(19) United States
(12) Patent Application Publication
(10) Pub. No.: US 2026/0207075 A1
(43) Pub. Date: Jul. 23, 2026

(54) Non-Contact Diagnostic System For Respiratory Disease Prognosis Through Thermal-Depth Spatiotemporal Behavioral Modeling

(71) Applicant: The Regents of the University of Colorado, a body corporate, Denver, CO (US)

(72) Inventors: Min-Hyung Choi, Superior, CO (US); Shane Transue, Arvada, CO (US)

(73) Assignee: The Regents of the University of Colorado, a body corporate, Denver, CO (US)

(21) Appl. No.: 19/142,772

(22) PCT Filed: Jan. 2, 2024
(86) PCT No.: PCT/US2024/010014

§ 371 Date: Jun. 24, 2025

Related U.S. Application Data
(60) Provisional application No. 63/478,082, filed on Dec. 30, 2022.

(51) Int. Cl. A61B 5/087; A61B 5/00; A61B 5/01; A61B 5/113; A61B 7/00; G16H 50/20

(52) U.S. Cl. CPC A61B 5/0873; A61B 5/0035; A61B 5/0075; A61B 5/01; A61B 5/0878; A61B 5/113; A61B 5/7267; A61B 5/7282; A61B 7/003; G16H 50/20; A61B 2576/00

(57) ABSTRACT

Systems and methods for respiratory analysis are disclosed herein. An implementation of the system includes a thermal imaging device that is configured to produce a time-based series of images representing gaseous flow indicative of pulmonary function. The system also includes an infrared depth imaging device configured to produce a time-based series of 3D point-cloud images of the monitored environment and subject. The system also includes a microphone that can produce a time-based sound recording. The system additionally includes a processing system that is configured to process the series of images, the series of 3D point-cloud images and the sound recording to produce a representation of exhalation flow, and a machine-learning model configured to produce an indication of a respiratory irregularity based on the exhalation flow.

### RELATED APPLICATIONS

This application hereby claims the benefit of and priority to International Application No. PCT/US2024/010014, titled “NON-CONTACT DIAGNOSTIC SYSTEM FOR RESPIRATORY DISEASE PROGNOSIS THROUGH THERMAL-DEPTH SPATIOTEMPORAL BEHAVIORAL MODELING,” filed Jan. 2, 2024; which claims the benefit of and priority to U.S. Patent Application No. 63/478,082, titled “NON-CONTACT DIAGNOSTIC SYSTEM FOR RESPIRATORY DISEASE PROGNOSIS THROUGH THERMAL-DEPTH SPATIOTEMPORAL BEHAVIORAL MODELING,” filed Dec. 30, 2022, which are hereby incorporated by reference in their entirety.

### STATEMENT REGARDING FEDERALLY SPONSORED RESEARCH

This invention was made with government support under grant number 1739452 awarded by the National Science Foundation. The government has certain rights in the invention.

### TECHNICAL FIELD

Aspects of the disclosure are related to the field of healthcare and health monitoring, and more particularly to the field of respiratory monitoring and diagnosis.

### BACKGROUND

Accurate diagnosis of pulmonary diseases is critical to effective treatment. In many cases, delayed diagnosis of pulmonary disease can result in significant increases in cost and time for treatment. Further, in many cases, delay of diagnosis can result in permanent damage.

An example of this is COPD caused by smoking. The damage caused by smoking builds up over many years before it can be detected through diagnostics, such as spirometry or plethysmography. By the time spirometry or plethysmography can detect the damage, the subject has typically progressed into the more severe stages of COPD, and much of the damage is irreversible.

Existing Pulmonary Function Tests (PFTs) do not allow testing on natural breathing. Rather, these PFTs require a modification of natural breathing, such as through breath restriction (such as requiring breathing through a tube), or requiring fixed quantity or concentration outputs. Natural breathing presents subtle traits and reoccurring abnormalities that are not likely to be present in such tests. A quantitative evaluation of natural breathing would present a better representation of subtle respiratory abnormalities that can be linked to specific pulmonary conditions.

### Overview

A respiratory diagnostics system for pulmonary condition analysis is disclosed herein. The system includes a thermal imaging device that is configured to produce a time-based series of images representing gaseous flow indicative of pulmonary function. The system also includes an infrared depth imaging device configured to produce a time-based series of 3D point-cloud images of the monitored environment and subject. The system also includes a microphone that can produce a time-based sound recording. The system additionally includes a processing system that is configured to process the series of images, the series of 3D point-cloud images and the sound recording to produce a representation of exhalation flow, and a machine-learning model configured to produce an indication of a respiratory irregularity based on the exhalation flow.

A respiratory diagnostics training system is also disclosed. The training system includes a CO2 filtered thermal depth imaging camera, a microphone, and memory device, and a processor. The memory device is configured to store a 3D density-flow representation from the depth imaging camera, sound data from the microphone, and a machine learning model. The processor is configured to identify a portion of the 3D density-flow representation that corresponds to an exhalation from a subject. The processor further correlates the 3D density-flow representation with some of the sound data. The processor is able to process the 3D density-flow representation, the sound data, and known pulmonary conditions into the machine learning model to create an updated machine learning model and store the updated machine learning model on the memory.

An additional respiratory diagnostics system is also disclosed. The system includes a CO2 filtered thermal depth imaging camera, a microphone that produces sound data that is correlated with data from the camera, a memory device configured to store a pulmonary characteristic database, and a processor. The processor is able to process correlated samples from the CO2 filtered thermal depth imaging camera and the microphone to identify a characteristic in the samples that corresponds to a pulmonary characteristic in the pulmonary characteristic database and flag the subject for a respiratory diagnosis.

### BRIEF DESCRIPTION OF THE DRAWINGS

Many aspects of the disclosure may be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 illustrates a camera in an example implementation.

FIG. 2 illustrates a training process in an implementation.

FIG. 3 illustrates a modeling system.

FIG. 4 illustrates a device network in an implementation.

FIG. 5 illustrates an analysis process in an implementation.

FIG. 6 illustrates a training process in an implementation.

FIG. 7 illustrates a modeling process in an implementation.

FIG. 8 illustrates a diagnostic process in an implementation.

FIG. 9 illustrates an operational sequence in an implementation.

FIG. 10 illustrates a computing system suitable for implementing the various operational environments, architectures, processes, scenarios, and sequences discussed below with respect to the Figures.

### DETAILED DESCRIPTION

Technology disclosed herein relates to systems and methods for diagnosing pulmonary conditions based on natural breathing. In particular, systems and methods are presented to translate images into predictions of gaseous fluid flow and volume according to standard units. Further, systems and methods are presented to create a database of breathing characteristics that can be compared to predictions of flow to diagnose pulmonary conditions.

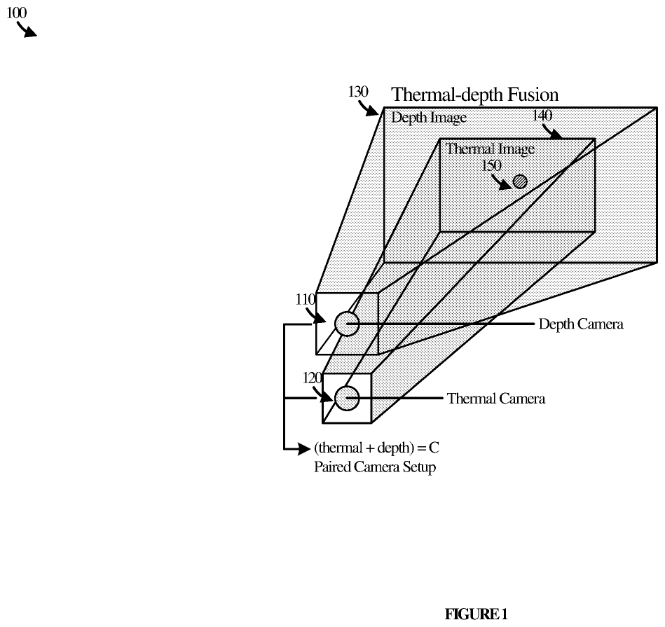

In an implementation, natural breathing can be analyzed through photographic data. This can be done, for example, by photographing exhaled CO2 from a subject. In FIG. 1, a system 100 is shown in accordance with an implementation. Depth camera 110 can be a camera capable of sensing depth. This could be, for example, a stereo vision camera, a time-of-flight camera, or a structured light camera, for example. Thermal camera 120 can be, for example, an infrared camera that is capable of creating an image based on the amount of heat in an object. The images from thermal camera 120 can be filtered to particular wavelengths in order to more closely define the images that are captured. Depth camera 110 and thermal camera 120 each have a field of view. The fields of view 130 and 140 shown in FIG. 1 differ slightly in size, but one of ordinary skill in the art should recognize that the fields of view 130 and 140 could also directly coincide.

Depth camera 110 and thermal camera 120 can be physically separate units, or they can be combined in one apparatus. Similarly, the images produced by depth camera 110 and thermal camera 120 can be separate, or a combined camera may internally process each of the images and output a combined thermal-depth image. In an implementation, depth camera 110 and thermal camera 120 produce images repeatedly, such as video images, such that a series of time-based images is produced.

Object 150 is shown in the thermal field of view 140 and the depth field of view 130. In an implementation, object 150 is captured in a set of time-based thermal images by thermal camera 120, and a corresponding set of time-based depth images by depth camera 110. These images can be combined to create a time-based set of 3D thermal images.

In an implementation, object 150 is an individual, particularly, an individual's head. Thermal camera 120 and depth camera 110 captures time-sequenced images of the individual's head as the individual breathed. The thermal images are filtered such that they display exhaled CO2 from the individual's mouth and nose. The sets of images from thermal camera 120 and depth camera 110 are combined to create a series of 3D thermal images that show CO2 exhaled by the individual.

This series of 3D thermal images can be converted into vectors representing the flow of CO2 in the exhalation. The images also contain magnitude information which can be associated with the flow vectors. In an implementation, this magnitude and flow information can be combined to create a model of the flow of CO2 being exhaled by the individual.

In an implementation, this model of flow can be correlated with actual known flow metrics. Many currently used pulmonary diagnostic tests use known metrics. By correlating the exhaled CO2 flow model with standard metrics, diagnostics can be enhanced. In an implementation, breath activity can be simultaneously measured by a currently available flow measurement, such as spirometry, and by the thermal camera 120 and depth camera 110 currently being described. After processing, the CO2 flow model of exhaled CO2 can then be calibrated with the other measured (e.g., spirometry) data. This could be a simple correlation, or it could be a much more complex machine learning process that incorporates the flow vector and magnitude data calculated from the thermal and depth images in order to create predictions of flow that correspond closely to the other measured (e.g., spirometry) data. The result of this calibration is that the data produced by the thermal camera 120 and depth camera 120 can be converted into estimations or predictions of flow metrics for the individual being photographed by the cameras. These flow metrics could include, for example, volume, volumetric flow, and velocity, among others.

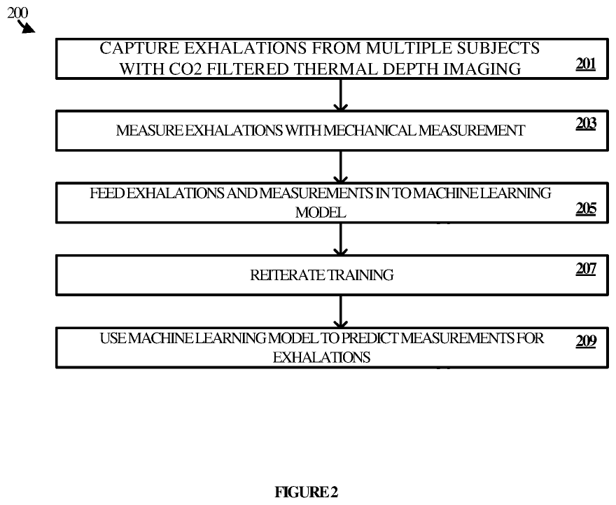

FIG. 2 shows a method of calibrating a CO2 flow model from thermal and depth images to measured flow metrics. In step 201, Exhalations are captured from multiple subjects. As discussed above, the thermal camera 120 and depth camera 110 can be used to record images of an individual exhaling. This same process can be used for multiple subjects, creating a collection of time-sequenced images corresponding to various subjects'exhalations. These time-sequenced images can be converted into a collection of CO2 flow models. In an implementation, each subject is recorded for several exhalations in order to create a large sample size. It should be noted that the data recorded from the subjects can include any combination of 1D, 2D and 3D data.

While the exhalations are being recorded, they are also measured directly with a spirometer in step 203. Various adjustments can be made in order to account for changes in the signal due to the presence of the spirometer. For example, the air can be heated such that is exits the spirometer at or near the temperature it would have been at the exit from the subject's mouth or nose. Following these measurements, a collection of CO2 flow models is stored, together with corresponding spirometry measurements. In an implementation, an alternate measurement of respiration such as plethysmography or capnography may be used in place of the spirometer.

In step 205, this data, or some of this data, is fed into a machine learning model. The machine learning model can be configured to correlate the flow vectors and magnitude information from the thermal and depth images with the velocity and volume measurements from the spirometer. The samples of data fed into the machine learning model can be selected to ensure that high quality data is used. In an implementation, the machine learning model can be used to evaluate breathing from multiple different subjects, with many breaths from each subject. This can allow for a correlation that provides reasonable accuracy across a large segment of the population. In another implementation, the machine learning model can be used to evaluate breathing over many breaths from a single individual. In this way, higher accuracy can be achieved for that particular individual. Step 207 shows that the machine learning can be iterated as many times as preferred. In some cases, a machine learning model that has already been put to use can be further refined by adding more patients and/or breath cycles as input to the model.

In step 209, the machine learning model can be used to process image data from depth camera 110 and thermal camera 120 to estimate or predict flow metrics for a patient in fields of view 130 and 140. Thus, by creating images for a patient, a prediction can be made for flow, volume and/or velocity that corresponds to a similar measurement that would be made using traditional measurement, such as a spirometer. This allows for measurement of natural breathing, as the subject can be recorded in any position, and for any amount of time, allowing for relaxation. In various implementations, a variety of metrics, such as breathing rate, flow, breathing effort or exhale volume and the distribution between nose and mouth breathing can be determined. In an implementation, no additional breathing obstruction needs to be added to the airflow for measurement.

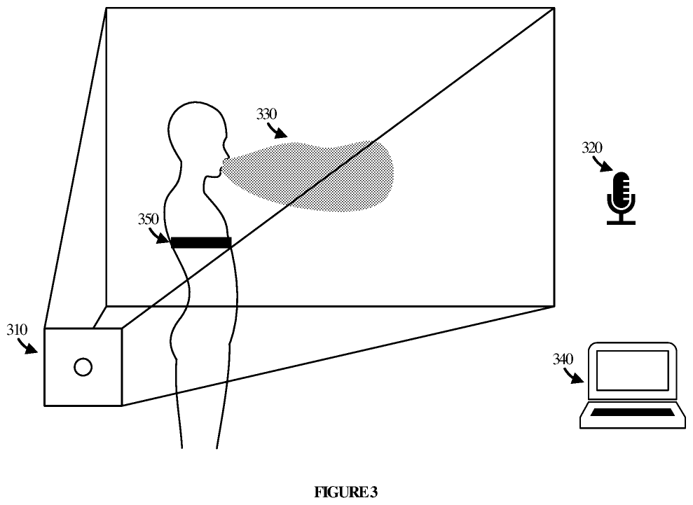

FIG. 3 shows a diagnostic system for diagnosing pulmonary conditions. Camera 310 is a combined depth/thermal camera, as discussed above. In an implementation, camera 310 is able to directly output predicted flow metrics as described above. In another implementation, camera 310 is able to output thermal and depth images which can be processed to produce flow metrics as discussed above. FIG. 3 shows a single combined thermal/depth camera 310, but distinct thermal and flow cameras could be implemented. Further the diagnostic system could use multiple cameras 310, either from similar orientations, or from differing orientations, in order to collect additional data.

Microphone 320 is shown as a single microphone but could similarly be an array of microphones. In an implementation, microphone 320 is correlated with camera 310, such that the data that is collected from camera 310 and microphone 320 is correlated. This correlation could be through the use of time stamps, file integration, or some other way.

Flow 330 is shown in the field of view of camera 310. This could be an exhalation from a human or animal subject as shown, or any other source of gaseous fluid flow. As discussed above, camera 310 is configured to produce flow data related to flow 330. This flow data can similarly be correlated with the data from microphone 320. Chest sensor 350 provides another source of data potentially related to the flow. Chest sensor 350 can be, for example, one or more transducers and/or accelerometers configured to measure chest movement and/or deformation. These sensors may be incorporated into a chest strap, for example. In some cases, chest sensor 350 may provide some interference to normal breathing. In other implementations, the chest sensor 350 may not interfere with normal breathing. The data from camera 310, microphone 320, and chest sensor 350 is transmitted to computer 340. Computer 340 contains a processor and memory. Computer 340 could be any type of computer, such as a personal computer, laptop, server, smart phone, specialized computer, etc. Computer 340 is configured to process the data from camera 310, microphone 320 and chest sensor 350.

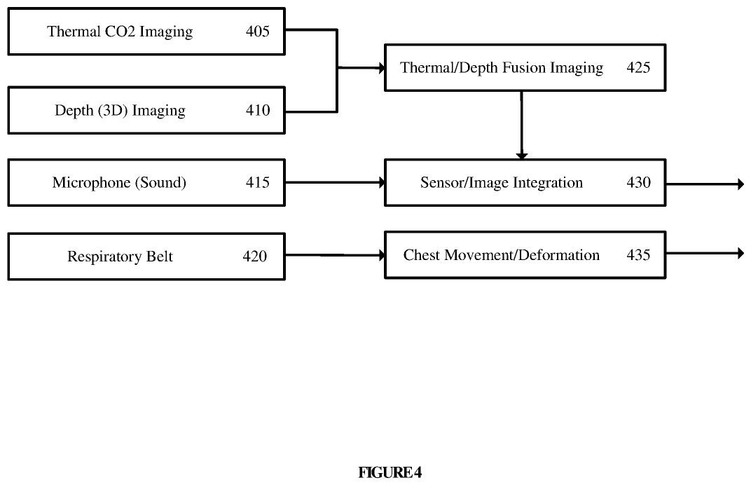

FIG. 4 illustrates an implementation of a processing progression of a diagnostic system. Element 405 represents thermal imaging. In an implementation, this imaging is done using a thermal camera which captures heated airflow. For example, a thermal camera can be used to capture breath (warm and primarily CO₂) leaving a subject. The subject could be a human subject or some other subject, such as an animal. In an implementation, this measurement could take place on a non-living subject. While an image is discussed, it should be understood that this discussion also applies to video recording or a plurality of images.

A 3D depth image is collected in element 410. This can be performed by any type of 3D or depth camera that can capture depth images. In an implementation, a depth image that will allow for interpretation of density of a gas cloud is collected. As with the thermal image discussed above, while an image is discussed, multiple images or video data can be collected in various implementations.

The thermal images and 3D depth images are combined in element 425. In an implementation, this combination is accomplished through the use of a camera that collects both thermal and 3D depth images at the same time and directly. In an implementation, element 425 results in a time-based series of images that provide a 3D representation of the exhalations of an individual.

In element 415, sound data is collected. In an implementation, this sound data is correlated to the time-based thermal and depth images of patient exhalations. The sound data can indicate breathing patterns or irregularities of the patient. In an implementation, this sound data is recorded by a microphone or a network of microphones.

In element 430, the sound data is integrated with the fused image data produced by element 425. In effect, this combination in element 430 can produce a stream of sound and image data, such as thermal depth video synchronized with sound. While FIG. 4 shows a certain order of combination to result in the sound and thermal depth video, one of ordinary skill in the art would understand that these pieces of data could be combined in many different ways to result in the described fused data. For example, a video camera configured to record thermal and depth images may combine all of the data directly in the camera, providing the thermal depth video as a direct output from the camera. Similarly, the respiratory belt data, discussed below, can also be combined with the thermal depth video data at any point in the process.

In element 420 movement data is collected from a respiratory belt. The respiratory belt may include transducers, motion sensors, accelerometers, pressure sensors, deflection sensors, continuity sensors, or other sensors to determine breathing motion or effort. While a respiratory belt or chest sensor belt is discussed herein, the sensors may not be mounted on a belt. The sensor may be individually mounted directly on the chest, or my sense chest movement from a distance, for instance. In element 435, the data from the respiratory belt can be used to produce a time-based waveform indicating chest movement of deformation.

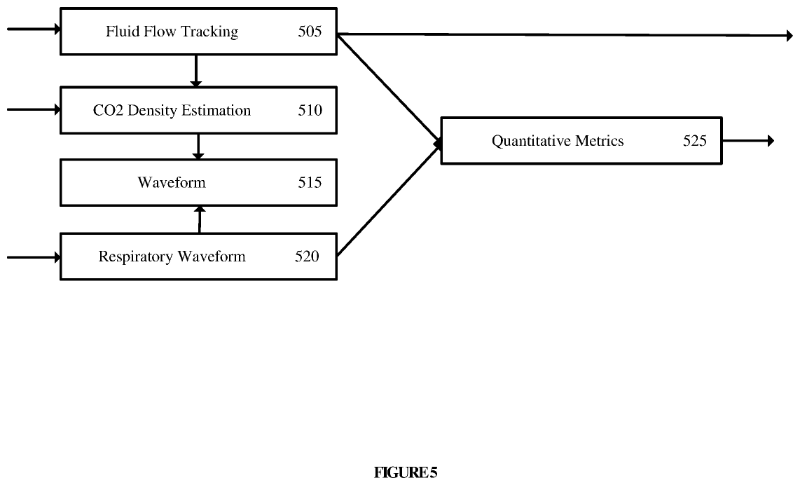

FIG. 5 illustrates an implementation of converting recorded data (such as the thermal depth image data, the sound data and the chest movement data collected in FIG. 4) into respiratory data. In element 505, an incoming signal is processed to produce fluid flow tracking information. For example, the input signal may be an integrated thermal depth video signal as discussed above. The time-based progressive images can be converted into vector data showing movement of images within the thermal depth video. In an implementation, various filters can be used to enhance the images of CO₂ being exhaled by the subject. The movement of the CO₂ is then converted to vector data, indicating flow of exhaled CO₂ over time.

In element 510 a CO₂ density estimation is determined. In an implementation, the integrated thermal depth video signal produced in FIG. 4 is processed to determine the density of the breath cloud exhaled by a patient. The fluid flow vectors determined by element 505 may also be used as inputs for this determination. In an implementation, the thermal depth video is converted to a representation of the breaths exhaled by a patient over time. This representation can include CO₂ density information and can provide a foundation to determine actual physical characteristics of fluid exhaled.

The CO₂ density data can be converted into a waveform in element 515. In an implementation, this waveform can represent the exhalations of a patient. This data can be consistent over time, such that if a patient increases or decreases actual exhalation volume, for example, the waveform can indicate the increase or reduction. In an implementation, the initial waveform does not indicate the actual units for measurement of these characteristics. For example, while the initial waveform can indicate that the exhalation volume of a particular patient increases over time, the waveform may not be able to indicate how the exhalation volume of one patient compares to the exhalation volume of another patient in another setting. Alternatively, the waveform produced may include absolute values, allowing comparison between patients.

In an implementation, the waveform can be produced by multiple methods. For example, in one method, the input signals can be mathematically analyzed to produce a waveform. The algorithm for this mathematical analysis can be manually created for this purpose. In a second method, a machine learning algorithm can be used to analyze the input signals and produce the waveform. In some implementations, a combination of these methods may be used.

In element 520, a respiratory waveform is created that can be used as an input to element 515. For example, the chest movement/deformation data produced in FIG. 4 can be converted into a waveform indicating respiration of a subject. This respiration waveform can be used together with fluid flow vectors and CO₂ density information to create the waveform of element 515.

The data produced by elements 505-520 can then be used to produce actual absolute value data for the patients. For example, using either analytical or machine learning processes, or some combination, the visual and other data collected from the subject can be compared with measured respiration data, such as spirometry data, to develop a conversion from unitless patient respiration data to absolute respiration data. After this conversion is identified, future unitless respiration data (such as that produced from the thermal depth images described herein) can be converted to absolute respiration data without the need for spirometry measurements. In an implementation, this conversion may allow for the identification of many metrics without needing a direct spirometry measure. For example, exhale velocity, exhale volume, nose-mouth exhalation distribution and breathing effort or strength may all be measured by this method.

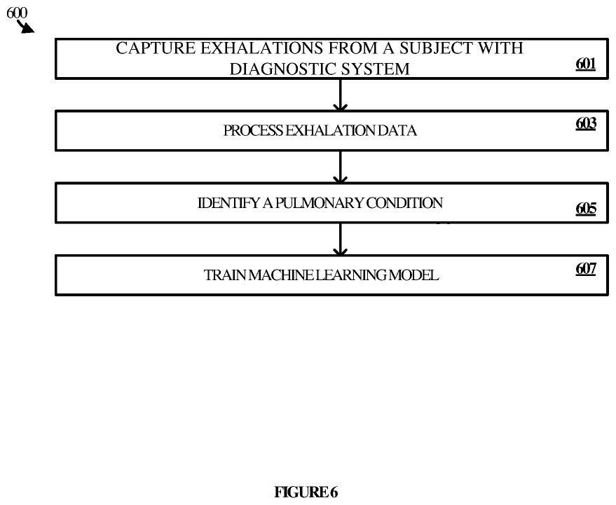

FIG. 6 illustrates training for a machine learning process in an implementation. In Step 601, exhalations from a subject are captured with the diagnostic system. In an implementation, this includes capturing combined thermal depth images, converting those images to flow metrics (Step 603), capturing sound data from microphone 320, and capturing measurements of chest movement and/or deformation from chest sensor 350. In an implementation, this could be a single breath cycle. In an alternate implementation, several breath cycles are captured and stored. This captured data is provided to computer 340. In addition to the captured data, known data is entered for the subject in step 605. In an implementation, this may occur through direct entry through a user interface for computer 340. In another implementation, this could occur through provision of a subject's electronic health record. This known data can include pulmonary irregularities for the subject, such as diagnosed pulmonary diseases. Additionally, the known data can include other known data for the subject, such as height, weight, race, gender, age, etc.

In an implementation, steps 601-605 are repeated for many subjects, with a variety of known data. In step 607, this data is then fed into a machine learning algorithm that will identify characteristics of exhalation flow that correspond to various diagnosed pulmonary diseases. One of ordinary skill in the art will recognize that various settings and inputs can be used to achieve a variety of outputs. For example, by feeding data from more subjects into the machine learning model, the likelihood of finding correlations increases. Additionally, by requiring a high degree of correlation, the number of identified potential diagnoses is reduced, but the likelihood of a false positive result is reduced.

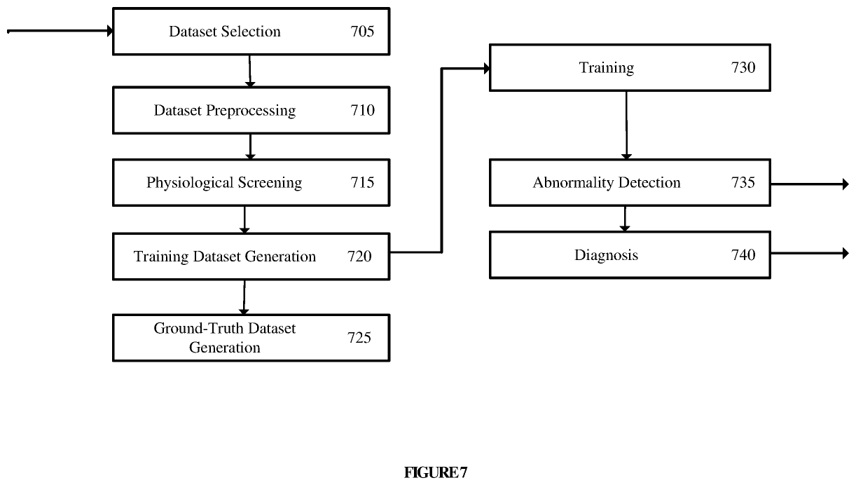

FIG. 7 illustrates an implementation of using the respiration data discussed above to detect and/or diagnose respiratory abnormalities. In an implementation, the diagnostic modeling in FIG. 7 is created by a machine learning algorithm with a variety of manual inputs.

In element 705, a dataset is selected. As discussed with regard to the Figures above, in an implementation, thermal depth image data, sound data and chest movement data is collected for a variety of subjects over time. This recorded data can be analyzed to identify data that is of poor quality or otherwise abnormal. This poor quality or abnormal data can be excluded from the potential dataset. Likewise, high quality data, such as subject data that includes a sufficient amount of clear data for a particular subject, may be included in the dataset. In an implementation, the quantitative metrics identified in FIG. 5 may also have a confidence factor that indicates how confident the algorithm is that the metrics are correct. Patient data that exceeds a threshold for the confidence factor may be included in the dataset. This dataset is preprocessed in element 710, for example, to ensure uniformity of the data in the dataset.

In element 715, physiological data (age, condition, race, sex, etc.) corresponding to the subjects whose data is in the dataset can be used to screen the data in the dataset. For example, the dataset could eliminate data from subjects that are subject to a particular respiratory ailment, or under a certain age limit. Alternatively, the data could be separated into groups by the physiological data. In an implementation, the physiological data may remain attached to the data in the dataset for future analysis.

Element 720 produces the training dataset, which will be processed by the training algorithm. The ground-truth dataset is also produced by element 725. For example, known respiratory ailments or irregularities corresponding to subjects in the dataset may be included. The training dataset is then trained by the machine-learning algorithm in element 730. This process can be iterative and may require many iterations to achieve acceptable performance. In an implementation, the trained algorithm may be able to identify irregularities or abnormalities in respiratory data. Further, it may be able to identify likely respiratory diseases or risks.

After training, in an implementation, elements 735 and 740 can detect abnormalities in respiratory data and make diagnoses for patients according to these abnormalities. It should be understood that in an implementation, the trained algorithm may be able to process new data (such as the data discussed with regards to FIGS. 1-5 above) to detect respiratory abnormalities and diagnose new patients.

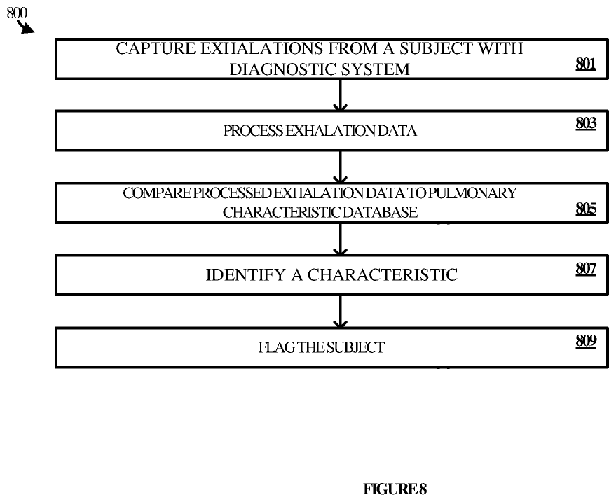

FIG. 8 illustrates a method of diagnosis of a pulmonary condition. In an implementation, this method uses the results of the machine learning model illustrated in FIGS. 6 and/or 7. In step 801, exhalations are captured from a subject. In an implementation, the diagnostic system shown in FIG. 3 is used to capture these exhalations. One of ordinary skill in the art will be able to select a useful number of exhalations to use for this diagnostic exercise. In an implementation, the captured exhalation data includes a time series of thermal and depth images, and a time-based audio signal. In Step8503, the data is processed. In an implementation, this processing includes translating the sequential thermal and depth images into a 3D density-flow representation. This could be, for example, the conversion to flow metrics as discussed above. Additionally, the processing could include correlating the audio data with the image or flow data.

In step 805, this processed data is compared to a pulmonary characteristic database. In an implementation, this pulmonary characteristic database is the output of the machine learning algorithm discussed above with regard to FIGS. 6 and/or 7. According to another implementation, this pulmonary characteristic database could be a conglomeration of known respiratory characteristics that correspond to pulmonary diseases. This database can include one or more characteristics of breathing that indicate that a subject may have a pulmonary disease.

By analyzing the data from the subject's exhalations, a characteristic of breathing can be identified that corresponds with the pulmonary characteristic database. In some cases, no characteristics will be found that correspond to the database.

If a characteristic is identified that corresponds to a pulmonary disease in the database, computer 340 can flag the subject. This could involve a display on a user interface on computer 340, a signal, such as a light or a sound, an entry to the subject's electronic health record, a text or email, or some other type of notification.

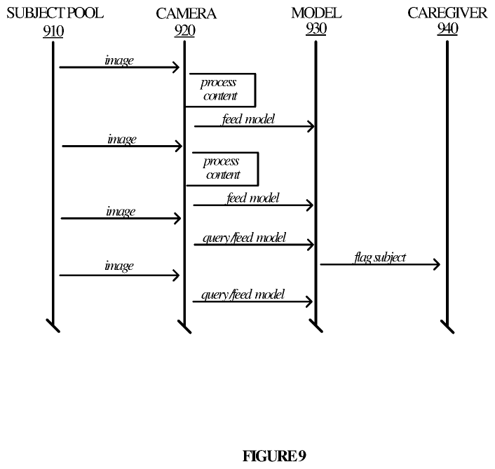

FIG. 9 illustrates an operational sequence according to an implementation. In the implementation, camera 920 is a combined thermal/depth camera. Camera 920 may also include a microphone and or chest belt as discussed above. Camera 920 records data of a set of images of a subject from subject pool 910, which include exhalation. This exhalation is made during natural breathing, and may occur standing up, sitting down, laying down, or in any other position. The exhalation may occur through subject's 910 mouth or nose.

Camera 920 then processes the image data. This processing may occur directly on the camera or may occur on an external processing device. The image data comprises a time series of thermal and depth images. These images are processed to produce magnitude and flow vector information. This magnitude and flow vector information is further used to estimate flow metrics. It should be understood that the processing could proceed directly from image data to flow metrics, without the intermediate step of calculating magnitude and flow vectors. Similarly, it should be understood that the image data may be directly recorded as 3D thermal images. It should further be understood that the processing could proceed directly from image data to pulmonary condition classifications and respiratory metric prediction through the machine learning model.

The flow metrics are entered into a machine learning model 930, together with known characteristics of the subjects. This machine learning model 930 will be understood to allow for the introduction of flow metrics for multiple subjects, and multiple breath cycles for each subject. Machine learning model 930 processes the data that has been provided to associate breathing characteristics with known pulmonary diseases. The machine learning model 930 can additionally account for age, race, gender, weight, or any other known subject characteristics provided.

Image data is then captured by camera 920 for another subject from subject pool 910. The data is processed and fed into machine learning model 930. It should be noted that the data fed into machine learning model 930 may differ slightly from that provided for another subject. For example, age and weight data may be known for a first subject, but not for a second. A first subject may include image data for 24 breath cycles, while another subject may only provide image data for 10 breath cycles.

After the machine learning model 930 has processed subject data sufficiently to associate breathing characteristics with pulmonary diseases, another subject from subject pool 910 provides image data to camera 920. In an implementation, the camera may feed image data directly to the machine learning model 930 if the model has been trained to take data directly from image data. In other cases, the camera will process the image data as shown and discussed above. The camera 920 then sends the data to the machine learning model 930 and queries the machine learning model 930. While this is shown as a query from the camera, it could also be a query from the computer, or some other source. The machine learning model 930 analyzes the image data to determine whether the subject has breathing characteristics that correlate with a pulmonary disease. If the subject does have breathing characteristics that correlate with a pulmonary disease, the patient will be flagged. This flagging may be a communication to a healthcare provider 940, a communication directly to the subject, or to some other target.

While the machine learning model 930 analyzes the data for this subject, the machine learning model 930 may incorporate the image and other data for subject into the model or not. This can be selected by the healthcare provider or equipment designer.

Further, the camera 920 may capture image data from a subject from subject pool 910, process the data and provide it to machine learning model 930. Machine learning model 930 may not find any breathing characteristics that indicate a pulmonary disease. Consequently, no flag is set. Again, the data from subject may be added to machine learning model 930 or not.

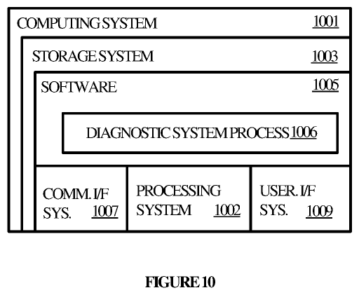

FIG. 10 illustrates computing system 1001 that is representative of any system or collection of systems in which the various processes, programs, services, and scenarios disclosed herein may be implemented. Examples of computing system 1001 include, but are not limited to, integrated circuits, SOCs, server computers, routers, web servers, cloud computing platforms, and data center equipment, as well as any other type of physical or virtual server machine, physical or virtual router, container, and any variation or combination thereof.

Computing system 1001 may be implemented as a single apparatus, system, or device or may be implemented in a distributed manner as multiple apparatuses, systems, or devices. Computing system 1001 includes, but is not limited to, processing system 1002, storage system 1003, software 1005, communication interface system 1007, and user interface system 1009 (optional). Processing system 1002 is operatively coupled with storage system 1003, communication interface system 1007, and user interface system 1009.

Processing system 1002 loads and executes software 1005 from storage system 1003. Software 1005 includes and implements diagnostic system process 1006, which is representative of the diagnostic processes discussed with respect to the preceding Figures. When executed by processing system 1002, software 1005 directs processing system 1002 to operate as described herein for at least the various processes, operational scenarios, and sequences discussed in the foregoing implementations. Computing system 1001 may optionally include additional devices, features, or functionality not discussed for purposes of brevity.

Referring still to FIG. 10, processing system 1002 may comprise a micro-processor and other circuitry that retrieves and executes software 1005 from storage system 1003. Processing system 1002 may be implemented within a single processing device but may also be distributed across multiple processing devices or sub-systems that cooperate in executing program instructions. Examples of processing system 1002 include general purpose central processing units, graphical processing units, application specific processors, and logic devices, as well as any other type of processing device, combinations, or variations thereof.

Storage system 1003 may comprise any computer readable storage media readable by processing system 1002 and capable of storing software 1005. Storage system 1003 may include volatile and nonvolatile, removable and non-removable media implemented in any method or technology for storage of information, such as computer readable instructions, data structures, program modules, or other data. Examples of storage media include random access memory, read only memory, magnetic disks, optical disks, optical media, flash memory, virtual memory and non-virtual memory, magnetic cassettes, magnetic tape, magnetic disk storage or other magnetic storage devices, or any other suitable storage media. In no case is the computer readable storage media a propagated signal.

In addition to computer readable storage media, in some implementations storage system 1003 may also include computer readable communication media over which at least some of software 1005 may be communicated internally or externally. Storage system 1003 may be implemented as a single storage device but may also be implemented across multiple storage devices or sub-systems co-located or distributed relative to each other. Storage system 1003 may comprise additional elements, such as a controller, capable of communicating with processing system 1002 or possibly other systems.

Software 1005 (diagnostic system process 1006) may be implemented in program instructions and among other functions may, when executed by processing system 1002, direct processing system 1002 to operate as described with respect to the various operational scenarios, sequences, and processes illustrated herein. For example, software 1005 may include program instructions for implementing a diagnostic system as described herein.

In particular, the program instructions may include various components or modules that cooperate or otherwise interact to carry out the various processes and operational scenarios described herein. The various components or modules may be embodied in compiled or interpreted instructions, or in some other variation or combination of instructions. The various components or modules may be executed in a synchronous or asynchronous manner, serially or in parallel, in a single threaded environment or multi-threaded, or in accordance with any other suitable execution paradigm, variation, or combination thereof. Software 1005 may include additional processes, programs, or components, such as operating system software, virtualization software, or other application software. Software 1005 may also comprise firmware or some other form of machine-readable processing instructions executable by processing system 1002.

In general, software 1005 may, when loaded into processing system 1002 and executed, transform a suitable apparatus, system, or device (of which computing system 1001 is representative) overall from a general-purpose computing system into a special-purpose computing system customized to establish connections and handle content as described herein. Indeed, encoding software 1005 on storage system 1003 may transform the physical structure of storage system 1003. The specific transformation of the physical structure may depend on various factors in different implementations of this description. Examples of such factors may include, but are not limited to, the technology used to implement the storage media of storage system 1003 and whether the computer-storage media are characterized as primary or secondary storage, as well as other factors.

For example, if the computer readable storage media are implemented as semiconductor-based memory, software 1005 may transform the physical state of the semiconductor memory when the program instructions are encoded therein, such as by transforming the state of transistors, capacitors, or other discrete circuit elements constituting the semiconductor memory. A similar transformation may occur with respect to magnetic or optical media. Other transformations of physical media are possible without departing from the scope of the present description, with the foregoing examples provided only to facilitate the present discussion.

Communication interface system 1007 may include communication connections and devices that allow for communication with other computing systems (not shown) over communication networks (not shown). Examples of connections and devices that together allow for inter-system communication may include network interface cards, antennas, power amplifiers, RF circuitry, transceivers, and other communication circuitry. The connections and devices may communicate over communication media to exchange communications with other computing systems or networks of systems, such as metal, glass, air, or any other suitable communication media. The aforementioned media, connections, and devices are well known and need not be discussed at length here.

Communication between computing system 1001 and other computing systems (not shown), may occur over a communication network or networks and in accordance with various communication protocols, combinations of protocols, or variations thereof. Examples include intranets, internets, the Internet, local area networks, wide area networks, wireless networks, wired networks, virtual networks, software defined networks, data center buses and backplanes, or any other type of network, combination of network, or variation thereof. The aforementioned communication networks and protocols are well known and need not be discussed at length here.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

The included descriptions and figures depict specific embodiments to teach those skilled in the art how to make and use the best mode. For the purpose of teaching inventive principles, some conventional aspects have been simplified or omitted. Those skilled in the art will appreciate variations from these embodiments that fall within the scope of the disclosure. Those skilled in the art will also appreciate that the features described above may be combined in various ways to form multiple embodiments. As a result, the invention is not limited to the specific embodiments described above, but only by the claims and their equivalents.

What is claimed is:

1. A respiratory diagnostics system for pulmonary condition analysis comprising:
a thermal imaging device configured to produce a time-based series of images representing gaseous flow indicative of pulmonary function;
an infrared depth imaging device configured to produce a time-based series of 3D point-cloud images of the monitored environment and subject;
a microphone configured to produce a time-based sound recording;
a processing system configured to process the series of images, the series of 3D point-cloud images and the sound recording to produce data representing exhalation flow;
a machine-learning model configured to produce an indication of a respiratory irregularity based at least in part on the data representing exhalation flow.

2. The system of claim 1 further comprising a chest sensor configured to produce an indication of chest movement, and wherein the machine-learning model further considers the indication of chest movement to produce the indication of the respiratory irregularity.

3. The system of claim 1, wherein the processing system transforms the time-based series of images and a first corresponding time-based series of 3D point-cloud images into data indicative of an exhalation density dissipation over time, and wherein the data representing exhalation flow comprises the data indicative of an exhalation density dissipation over time.

4. The system of claim 1, wherein the processing system is configured to use a machine-learning process to transform time-based the series of images, the series of 3D point-cloud images and the sound recording to produce data representing exhalation flow.

5. The system of claim 1, wherein the data representing exhalation flow comprises a flow velocity, a flow volume, an exhalation volume, a nose-mouth breathing ratio or a breathing effort.

6. The system of claim 4, wherein the machine-learning process is further configured to consider a spirometry measurement to produce the data representing exhalation flow.

7. The system of claim 1, wherein the machine-learning model is further configured to produce a respiratory diagnosis based at least in part on the data representing exhalation flow.

8. A respiratory diagnostics training system, comprising:
a CO2 filtered thermal depth imaging camera, having a camera field of view, and configured to produce a 3D density-flow representation for a gaseous flow within the camera field of view;
a microphone configures to produce sound data;
a memory device configured to store thereon the 3D density-flow representation, sound data from the microphone, and a machine learning model; and
a processor, configured to:
identify a first subset of the 3D density-flow representation corresponding to an exhalation from a subject, having a known pulmonary condition, and located in the camera field of view;
correlate the first subset of the 3D density-flow representation with a subset of the sound data;
process the first subset of the 3D density-flow representation, the subset of the sound data, and the known pulmonary condition into the machine learning model to create an updated machine learning model; and
store the updated machine learning model on the memory.

9. The system of claim 8, further comprising a chest sensor configured to product chest movement data, and wherein the processor is further configured to correlate a second subset of the chest movement data with the first subset of the 3D density-flow representation and the subset of the sound data.

10. The system of claim 8, wherein the CO2 filtered thermal depth imaging camera comprises a CO2 filtered thermal camera and a depth camera, both configured to capture the camera field of view.

11. The system of claim 10, wherein producing a 3D density-flow representation for gaseous flow within the camera field of view comprises aligning a first set of images from the CO2 filtered thermal camera with a second set of images from the depth camera and processing the aligned sets of images into a set of spatial models of exhale behaviors.

12. The system of claim 11, further comprising processing the set of spatial models of exhale behavior into a spatiotemporal 4D density dissipation model of exhale behaviors.

13. The system of claim 8, further comprising a translation engine configured to translate first image data from the CO2 filtered thermal depth imaging camera into the 3D density-flow representation.

14. The system of claim 13, wherein the translation engine is part of the CO2 filtered thermal depth imaging camera.

15. The system of claim 13, wherein the translation engine is operated by the processor.

16. The system of claim 8, wherein the machine learning model is configured to identify a first pulmonary characteristic represented in the 3D density-flow representation that corresponds to a respiratory diagnosis.

17. A respiratory diagnostics system, comprising:
a CO2 filtered thermal depth imaging camera, having a camera field of view;
a microphone, wherein data from the microphone is correlated with data from the CO2 filtered thermal depth imaging camera;
a memory device configured to store thereon a pulmonary characteristic database; and
a processor, configured to:
process a first collection of correlated samples from the CO2 filtered thermal depth imaging camera and the microphone, which correspond to an exhalation of a subject within the field of view;
identify a characteristic in the first collection of correlated samples that corresponds to a first pulmonary characteristic stored in the pulmonary characteristic database, where the first pulmonary characteristic is associated with a respiratory diagnosis in the pulmonary characteristic database; and
flag the subject for the respiratory diagnosis.

18. A respiratory diagnostics system of claim 17, wherein the characteristic corresponds to one or more of a peak, a valley, a flow velocity, or a vortex of turbulent flow correlated with the exhalation of the subject.

19. A respiratory diagnostics system of claim 17, wherein processing the first collection of correlated samples from the CO2 filtered thermal depth imaging camera and the microphone, further comprises identifying that the exhalation of the subject is an exhalation from the subject's mouth.

20. A respiratory diagnostics system of claim 17, wherein processing the first collection of correlated samples from the CO2 filtered thermal depth imaging camera and the microphone, further comprises identifying that the exhalation of the subject is an exhalation from the subject's nose.
